(19) United States
(12) Patent Application Publication
(10) Pub. No.: US 2005/0172965 A1
(43) Pub. Date: Aug. 11, 2005

(54) Breathing device

(76) Inventor: Bjorn Thulin, Stockholm (SE)

Correspondence Address: FASTH LAW OFFICES (ROLF FASTH), 26 PINECREST PLAZA, SUITE 2, SOUTHERN PINES, NC, 28387-4301, US

(21) Appl. No.: 10/514,521

(22) PCT Filed: May 22, 2003
(86) PCT No.: PCT/US03/16531

Related U.S. Application Data
(60) Provisional application No. 60/384,007, filed on May 28, 2002.

(51) Int. Cl. A62B 7/00; A61M 16/00

(52) U.S. Cl. 128/204.18

(57) ABSTRACT

The breathing device (10) has an inner section (14) having an inlet (32) defined therein. The section (14) has a motor (20), a fan (24) and a processor (22) disposed therein and the section (14) has an outlet (34) defined therein. The outlet (34) is in fluid communication with the inlet (32) so that pre-heated air is being be propelled by the fan (24) to pass from the inlet (32) across the processor (22), over the motor (20) and out through the outlet (34). An outer section (16) has a container (26) attached thereto that has a channel (42) defined therein. The channel (42) has a water-absorbing material (44) disposed therein for increasing the humidity of the air passing therethrough. A tube (28) is connected to the outer section (16) for carrying air from the channel (42) to a mask (30) attached to one end of the tube (28).

### TECHNICAL FIELD

The present invention relates to a breathing device that may be used to avoid undesirable snoring in general and obstructive sleeping apnea syndrome (OSAS) in particular and other breathing or air passage difficulties.

### BACKGROUND INFORMATION AND SUMMARY OF THE INVENTION

Snoring and the obstructive sleeping apnea syndrome (OSAS) are growing problems in today's society. Many people suffer from abnormal tiredness during the day due to inadequate sleeping. A particular problem is that the breathing during sleep while snoring is disturbed, so that the normal breathing may be interrupted for about 10 seconds up to 90 seconds. This may result in an undesirable decrease of the oxygen level in the blood of more than 4%. The inadequate sleeping may also lead to an increase of blood pressure, thus leading to heart and blood-vessel diseases and difficulties with memorization and concentration. Many types of devices have been developed to help patients breath better during sleep. For example, ventilators, so called CPAPs(continous positive airway pressure), have been developed that provide air under an overpressure into a mask that is mounted over the nose and sometimes over the mouth and nose. The ventilators keep the air passageways open to prevent snoring and other breathing difficulties. One major problem with the currently available ventilators is that the air provided is both too dry and cold that adversely affects the mucous membranes and lungs. This is a particular problem for certain categories of people such as people who suffer from allergy or asthma or who are singers or actors.

There is a need for an apparatus that prevent snoring, apneas and other breathing difficulties during sleep while maintaining the mucous membranes and lungs in good condition. The breathing device of the present invention provides a solution to the above-outlined problems. More particularly, the breathing device of the present invention has an inner section having an inlet defined therein. The section has a fan and a processor disposed therein. The inner section has an outlet defined therein. The outlet is in fluid communication with the inlet so that preheated air is being be propelled by the fan to pass through a biological microfilter from the inlet across the processor, motor and through the outlet. An outer section has a water filled container attached thereto that has a channel defined therein. The channel has a water-absorbing material as a tube disposed therein, thereby magnifying the absorbing area by, for example, up to about three times. An outer plastic tube is connected to the outer section for carrying air from the channel to a mask attached to one end of the tube. The breathing device both pre-heats and increases the temperature of the air that goes into the mask, thereby making the air possible to keep more humidity according to general physical laws of dew point.

### BRIEF DESCRIPTION OF THE DRAWINGS

FIG. 1 is a top view of the breathing device of the present invention;

FIG. 2 is an elevational side view along line 2-2 of FIG. 1;

FIG. 3 is a detailed top view of the humidifier device of the present invention; and

FIG. 4 is a lid for the humidifier device of FIG. 3.

### DETAILED DESCRIPTION

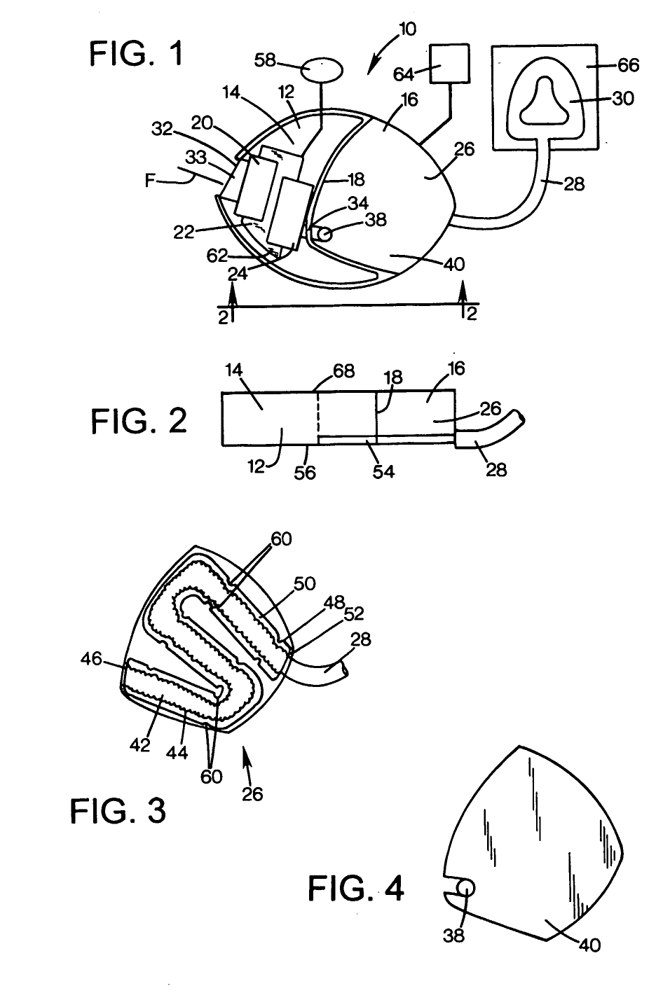

With reference to FIGS. 1-4, the breathing device 10 of the present invention has a boomerang formed housing 12 that has an inner section 14 and an outer trapeziformed section 16. A vertical wall 18 encloses the section 14. The section 14 has an electric motor 20 for driving a fan device 24 and an electronic microprocessor 22 is also disposed therein. The motor 20 may be integrated with the fan device 24 so that they form one piece. The section 16 is adapted to hold a humidifier device 26 (FIG. 3 with lid FIG. 4) that contains water. The housing 12 is through the humidifier device connected to a hose 28 that has a mask 30 attached at one end thereof. The mask 30 may be customized to the face of the user so that the mask is held in place without any elaborate fasteners.

An air intake 32 is defined in the inner section 14 so that air may flow according to an arrow F and into the section 14. The air intake 32 may have a biological micro-filter 33 disposed therein that filters out any undesirable articles, such as pollen and dust, in the incoming air. The air may be sucked in by the fan device 24 so that air passes the motor 20 and over the processor 22 to cool the motor and processor 22 before the fan device 24 propels the air out through an opening 34 and into a hose 36 that leads into an opening 38 of a lid 40 of the humidifier device 26.

The fan device 24 creates an over pressure that is 5 to 20 cm column of water (5-20 mBar) higher than the atmospheric pressure. The lid 40, as best shown in FIG. 4, is adapted to fit over the humidifier device 26, as best shown in FIG. 3. Because the air is passing the motor 20 and the microprocessor 22, the air is pre-heated by the warm or hot motor 20 and the processor 22. The device 26 is removable from the housing 12 so that it may be machine-washed or otherwise be cleaned in between use. The size of the device 26 should be such that the humidity provided by the evaporated water in the device 26 and the water soaked material should be sufficient to last at least one night for a user 66.

More particularly, the heated air is propelled from the opening 38 into an S-curved channel 42, as best shown in FIG. 3, that is defined in the humidifier device 26. A tube-formed material 44 saturated with water disposed in the channel 42. The water in the device 26 and the tube 44 is permitted to evaporate. Because the air passing therethrough is heated from about 20 C up to about 25 C, the dew point of the air is higher than the dew point at room temperature. This means the air may be more humid at the higher temperature. The air must pass through the entire device 26 from an inlet end 46 to an outlet end 48 of the channel 42 and be exposed to the evaporating water of the device 26 and the tube-formed material 44 that bears against the inner wall of the channel 42. The material 44 absorbs and maintains the water 50 that is disposed in the channel 42 and the ventilation air that is passed through the channel is exposed to a larger surface area of evaporating water compared to a plain water surface. The channel 42 has a plurality of holding sections 60 to hold the material 44 in place. The S-shape of the channel 42 creates a very large surface area of the material 44 in a relatively small volume. Because the air is exposed to the water saturated material 44, the humidity of the air is substantially increased before the air moves out through an opening 52 and into the tube 28 and the mask 30.

The further increase the temperature of the air, the housing 12 may have a heating film 54, best shown in FIG. 2, disposed between a bottom 56 of the housing 12 and below the humidifier device 26. The film 54 may increase the temperature of the air flowing through the device 26 another 3-4° C. Of course, the film 54 may be set to heat up the air more or less than 3-4° degrees C. By heating the film 54, the water in the device 26 is also heated by conduction so that the air flowing therethrough is also heated. The device 10 also has a top cover 68 to hold the device 26 in place when placed in the section 16.

The device 10 may also have a built in sensors 58 connected thereto for measuring the pulse, blood pressure, blood oxygen level and other such parameters of the user 66. The sensor 58 may be connected to the circuit board/processor 22. The processor 22 may have a communication module 62 that may communicate by wired or wireless technology with a central computer 64. The module 62 may provide information regarding the condition of the user 66 by collecting and analyzing the data from the sensor 58 and also the condition of the device 10. By being able to remotely analyze the condition of the user 66, there is less need to be bodily present to visit a medical professional. The processor 22 may also be used to regulate the speed of the fan device 24 and the temperature of the heating film 54 to make sure the pressure, temperature and humidity of the air passing through the tube 28 is correctly adjusted to the specific needs of the user 66.

The device 10 may also have sensors for measuring the outgoing air in order to automatically adjust pressure, humidity and temperature and other characteristics of the outgoing air. The device 10 may also control and monitor the ingoing air in a similar way.

While the present invention has been described in accordance with preferred compositions and embodiments, it is to be understood that certain substitutions and alterations may be made thereto without departing from the spirit and scope of the following claims.

1. A breathing device, comprising:
an inner section (14) having an inlet (32) defined therein, the section (14) having a fan (24) and a processor (22) disposed therein, the section (14) having an outlet (34) defined therein, the outlet (34) being in fluid communication with the inlet (32) so that air is being be propelled by the fan (24) to pass from the inlet (32) across a motor (20), the processor (22) and out through the outlet (34);
an outer section (16) attached to the inner section (14), the section (16) having a container (26) attached thereto, the container (26) having a channel (42) defined therein, the channel (42) having a water absorbing material (44) disposed therein; and
a tube (28) connected to the outer section (16) for carrying air from the channel (42) to a mask (30) attached to one end of the tube (28).

2. The breathing device according to claim 1 wherein the channel (42) is S-shaped.

3. The breathing device according to claim 1 wherein the container (26) has a lid (40), the lid (40) having an opening (38) defined therein for communicating with

4. The breathing device according to claim 1 wherein the inlet (32) has a filter (33) disposed therein.

5. The breathing device according to claim 1 wherein the breathing device (10) has a sensor (58) connected thereto.

6. The breathing device according to claim 1 wherein the processor (22) has a communication device (62) for wireless communication with a computer (64).

7. The breathing device according to claim 1 wherein the container (26) has a heating layer (54) disposed below the container (26) and above a bottom (56) of a housing (12).

8. The breathing device according to claim 1 wherein the material (44) are attached along side walls of the channel (42).
